(19) United States
(12) Patent Application Publication
(10) Pub. No.: US 2010/0185041 A1
(43) Pub. Date: Jul. 22, 2010

(54) Cylindrical magnetic field projection method

(76) Inventor: Richard H. Lee, Laguna Niguel, CA (US)

Correspondence Address: RICHARD H. LEE, 22 WOODHAVEN DRIVE, LAGUNA NIGUEL, CA, 92677, US

(21) Appl. No.: 12/321,242

(22) Filed: Jan. 20, 2009

(51) Int. Cl. A61N 2/02; H01F 7/06

(52) U.S. Cl. 600/13; 335/219

(57) ABSTRACT

A method for creating a cylindrical magnetic field with accompanying linear electric vector potential, and projecting it into a substance, and a method for modulating this field. In one embodiment the field is projected into a person for therapy with a circumferentially magnetized ferromagnetic cylinder. This field is then modulated with toroidally wound coil to carry the modulated signal deep into the body. A principal therapeutic benefit of this embodiment is rapid pain relief.

### CROSS-REFERENCE TO RELATED APPLICATIONS

One embodiment of this invention uses frequency bands of unpredictable signals and general method of inducing signals into people for therapy disclosed in my patents U.S. Pat. No. 6,461,316 granted Oct. 8, 2002, U.S. Pat. No. 6,770,042 granted Aug. 3, 2004, and U.S. Pat. No. 7,419,474, granted Sep. 2, 2008, which are incorporated by reference. While these specific signals are not claimed in this specification, this invention can be used to introduce these signals into people for therapy.

### BACKGROUND OF THE INVENTION

1. Field of Invention

This invention generally relates to methods to induce electric and magnetic fields into substances. Specifically, it describes a means for inducing generally cylindrical magnetic signals in substances. In one embodiment the cylindrical magnetic field is induced into a person for therapy with a circumferentially magnetized cylinder. This field is modulated with toroidally wound coil. A principal therapeutic benefit of this embodiment is rapid pain relief. More broadly, the invention is a method to induce a cylindrical magnetic field and linear electric vector potential in any substance.

2. Background of the Invention

Many methods have been used to induce signals into people for therapy. Many are effective, some more than others, and some excel for specific purposes. For instance massagers produce a spherically radiating acoustical wave, ultrasound produces local agitation, heating and micro-fluidity, magnetic pulse devices produce planar or spherical magnetic waves and electric stimulation, whether through the skin like TENS and EMS, or invasive electrical stimulation with implanted electrodes, produces electrical currents in the body. There are also many devices which broadcast electromagnetic signals into the body. All of these are substantially different than the current invention.

One way to deliver a signal is to establish a field in the body, then to modulate that field with a signal. As an example, one of the methods of prior art, U.S. Pat. No. 6,461,316, involves using a permanent magnet to produce a magnetic field that penetrates a person, and using a coil of wire to modulate this same magnetic field much like an acoustical speaker driver, modulating the magnetic field projected into the person, thereby inducing magnetic and electrical signals for therapy into the person's body. This is also an object of the invention disclosed here, except that, instead of projecting, into the body, a toroidally shaped magnetic field, which creates circular electrical potentials and circular magnetic currents, this invention projects a cylindrical magnetic field into the body and creates a linear electric vector potential which is much more linear than the curving magnetic and circular electric fields created in U.S. Pat. No. 6,461,316. This cylindrical magnetic field penetrates more deeply into the body. FIGS. 1-3 illustrate this important distinction between the prior art and the current method.

Many therapy devices involve magnetic fields such as PEMF devices as described in U.S. Pat. No. 6,955,642 and U.S. Pat. No. 6,443,883 However these do not induce cylindrical magnetic fields into the body.

U.S. Pat. No. 6,780,150 describes an annular coil, but it is a strip magnet rolled into a coil such that the magnetic field is radial to the annulus. U.S. Pat. No. 5,842,966 describes another coil but it provides a magnetic field which is axial to the annular coil. The present invention differs from both in that it provides a magnetic field which is circumferential to the cylindrical shape.

Several patents such as U.S. Pat. No. 6,239,760 and U.S. Pat. No. 4,751,515 describe toroidally wound coils, copper wire helically wound around toroidal shapes. In fact, toroidally wound coils are very common in electronic design as transformers and chokes. U.S. Pat. No. 6,361,810 discloses a toroidally wound coil influencing a substance it was placed next to by means of a “vector field, a field with no magnetic component.” Toroidally wound coils, while they do induce magnetic fields and electric vector potentials, do not produce a cylindrical magnetic field or a linear electric vector potential. While a toroidally wound coil is one of the elements of the current invention, it is used only to modulate a pre existing cylindrical magnetic field and a linear electric vector potential. In the current invention, a cylindrical magnetic field is produced by a circumferentially magnetized generally cylindrical ferromagnetic core, with height at least double the wall thickness, and preferably at least 10 times wall thickness, to provide a cylindrical magnetic field. This cylindrical shape causes the field to penetrate as a ray, deeply into tissue with the produced linear electrical vector potential supporting the projection of the cylindrical magnetic field through substances with magnetic qualities.

Thus, none of the prior art discloses the structure (circumferentially magnetized cylinders, linear electric vector potentials, or toroidal coils positioned axially to linear electric vector potentials). Nor does prior art disclose a cylindrical magnetic field or linear electric vector potential which is induced deeply into a person or substance. Nor does it disclose modulating such a field to induce a signal. The use of cylindrical magnetic fields and linear electric vector potentials has not heretofore been disclosed.

### SUMMARY

A method for creating a cylindrical magnetic field in a substance, and a method for modulating this field. In one embodiment the cylindrical magnetic field is induced into a person for therapy with a circumferentially magnetized cylinder. This field is then modulated with toroidally wound coil. A principal therapeutic benefit of this embodiment is rapid pain relief.

### SUMMARY OF THE INVENTION WITH OBJECTS

The objects and advantages of one therapeutic embodiment of this invention are: (a) to provide an improved therapy device, (b) to provide a device that will create a field that penetrates deeply into the body, (c) to engage the body electrically so as to make contact with electrical stagnation within the body, and (d) to induce a signal in the body through the created field to break up electrical stagnation. This invention succeeds at these objectives, and provides benefits such as fast pain relief. In addition, because it combines a high-strength fixed-magnet field generator with a low powered toroidally-wound coil field modulator, it provides a strong field and relatively low power draw, making it suitable for battery powered operation.

The cylindrical magnetic field can also be projected into other substances to induce linear electric and cylindrical magnetic alignments, and to induce signals. Further objects and advantages will become apparent from a consideration of the ensuing description and drawings.

### BRIEF DESCRIPTION OF THE DRAWINGS

FIGS. 1, 2, and 3 show three different magnetic field configurations, each producing a different distribution of lines of electric vector potential. These figures illustrate the cylindrical magnetic field of this invention and differentiate it from the prior art. FIG. 1 shows an axially magnetized bar magnet. FIG. 2 shows a circumferentially magnetized toroidal ring magnet. FIG. 3 shows a circumferentially magnetized thin wall cylindrical magnet.

FIG. 4 shows a circumferentially magnetized cylinder inducing a circumferential magnetization and linear electric vector potential in a ferromagnetic cylinder. This illustrates how a circumferentially magnetized cylinder might propagate through somewhat magnetic substances including air, water, and human body tissue. It also illustrates how adding a longer ferromagnetic tube might amplify the linear electric vector potential and make it more linear, increasing its reach.

FIG. 5 shows the principal embodiment of this invention with signal generator circumferentially magnetized cylinder, toroidally wrapped modulating coil, and substance into which the modulated linear electric vector potential field is projected.

### DETAILED DESCRIPTION OF THE DRAWINGS

FIG. 1 shows an axially magnetized cylindrical magnet 10, generating generally circular magnetic lines of force 12, and generally circular electric vector potentials 14. A longer magnet would tend to create more linear magnetic lines of force. While the magnetic field projects axially beyond the magnet, this axially magnetized structure 10 does not create linear electric vector potentials, but rather circular ones.

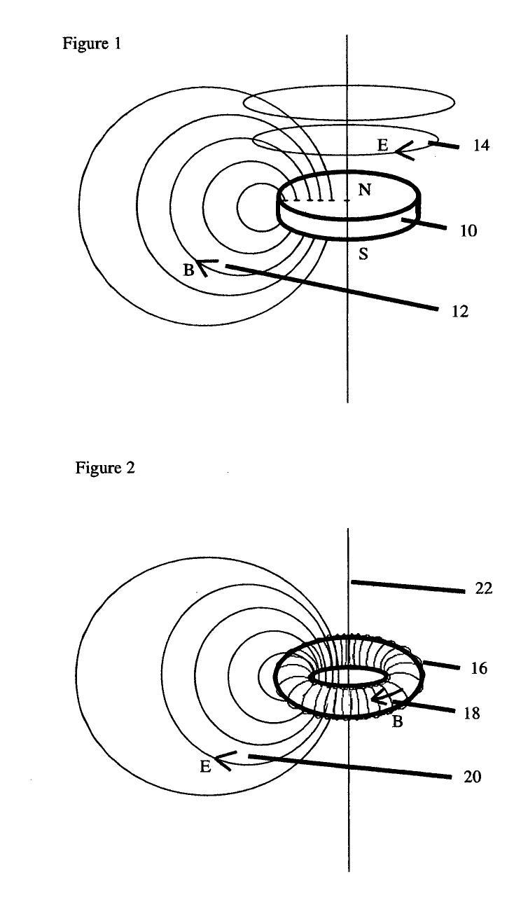

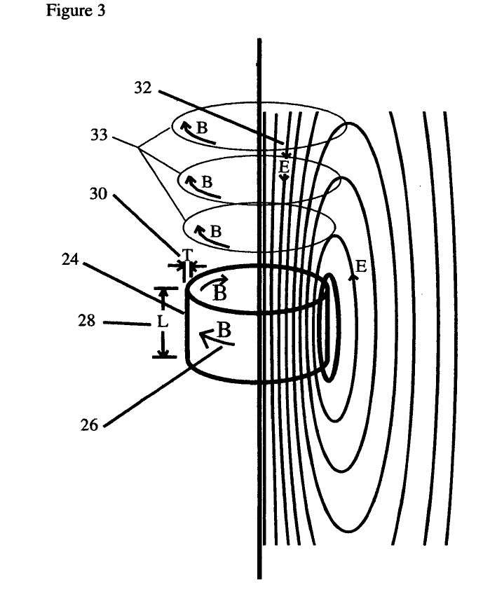

FIG. 2 shows a toroidal ring 16, magnetized circumferentially 18 with a strong cylindrical magnetic current flowing through the cross section around the circumference of the ring. This magnetic current creates generally circular lines of electric vector potential 20 with minimal electric field potential 22 projecting linearly in an axially direction.

FIG. 3 shows hollow cylinder 24 magnetized circumferentially 26. Because length 28 of hollow cylinder 24 is considerably greater than wall thickness 30, much of the electric vector potential 32 projects axially 34 for a great distance, mostly linearly. As the ratio of cylinder length 28 over wall thickness 30 increases, electric vector potential 32 becomes more linear and projects even further. The term linear electric vector potential is used to differentiate from the generally circular electric vector potential present in other therapy devices as illustrated in FIGS. 1 and 2. While in the first embodiment, length 28 is about ten times greater than wall thickness 30, the electric vector potential starts becoming more linear as the shape elongates from a toroid to a cylinder, say as length is twice the thickness. On the other hand, length can be 100 times wall thickness or more to provide greater linearity and reach. Cylindrical magnetization 33 is induced in air by the linear electric vector potential.

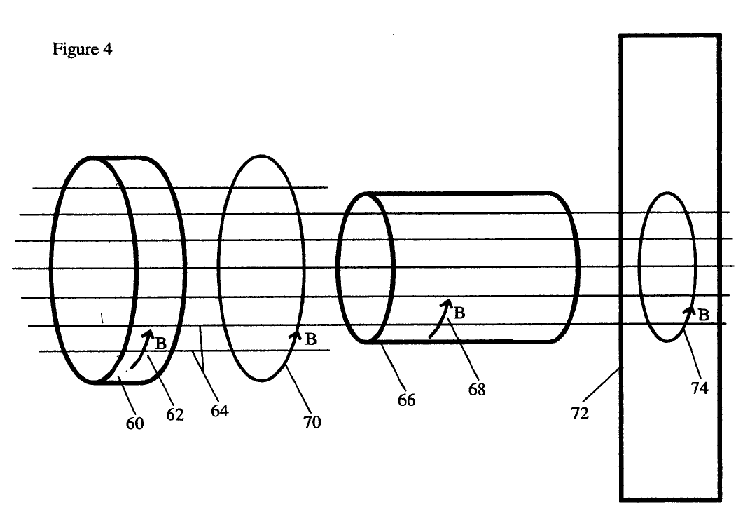

FIG. 4 shows primary circumferentially magnetized cylinder 60 with circumferentially magnetization 62, which creates linear electric vector potential 64 through secondary ferromagnetic cylinder 66, inducing secondary circumferential magnetization 68 in cylinder 66. As electric vector potential 64 travels between cylinder 60 and cylinder 66, potential 64 induces circular magnetization 70 in air, which supports potential 64. Then as potential 64 penetrates living tissue 72, potential 64 produces circular magnetization 74 in tissue 72, again supporting the projection of linear electric vector potential into tissue 72. This stable electromagnetic field configuration, with circular magnetic flux 74 around linear electric vector potential is what allows cylindrical magnetic field 74 and linear electric vector potential 64 to penetrate deeply into living tissue. While it appears that both the electric and the magnetic component are mutually supportive as they project into the living tissue, the invention can be described either as cylindrical magnetic field 74 or linear electric vector potential 64 projecting into the body. It is not clear which is the biologically active ingredient which induces health benefits such as pain relief Since both project into the body together, it appears the specific theoretical mechanism of bio effectiveness is unimportant.

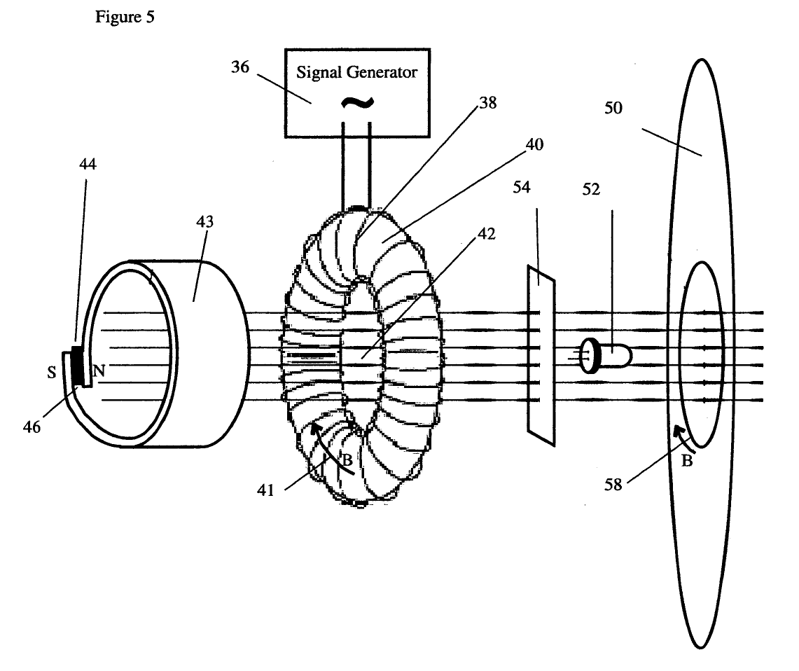

FIG. 5 shows a therapeutic embodiment of this invention. Signal generator 36 generates time varying electric current through magnet wire 38 which is toroidally wound around ferromagnetic ring 40, which produces a time varying magnetic current 41 through ring 40 in a circumferential direction, modulating linear electric vector potential 42 passing perpendicularly through the plane of ring 40, also passing through cylinder 43 and surface 50. Potential 42 is generated by, and axially to, circumferentially magnetized ferromagnetic hollow cylinder 43, which is magnetized by permanent magnet 44, inserted in gap 46 of hollow cylinder 43. The modulated linear electric vector potential 42 penetrates surface of living tissue 50, symbolized as a large oval, for therapy. This can also be described in terms of circumferential magnetic current as follows. Circumferentially magnetized ferromagnetic hollow cylinder 43 produces linear electric vector potential 42, which produces magnetic currents 58 in whatever magnetic substance linear electric vector potential 42 penetrates, whether air, which has magnetic oxygen molecules, water, which has magnetic water molecules or even space which contains a sea of neutrons, any of which can align to support a circular circulation of magnetic current that, in turn supports linear electric vector potential 42 as it penetrates air and human tissue. Magnetic currents 58 are shown as discrete loops for the sake of illustration, but in actuality are a continuous invisible magnetic cylinder extending from magnetic cylinder 43, and transitioning to slightly magnetic air, then to the slightly fluid in a human body 50.

A second means of modulating said linear electric vector potential 40 and providing therapy involves inserting photon source 52, a 630 nm LED into the linear electric vector potential. A third means of modulating said linear electric vector potential and providing therapy involves inserting mirror 54 such that it is perpendicular to linear electric vector potential 48 and reflects photons emerging from living tissue 50 back into living tissue 50. In the above, electric vector potential 56 generated by the cylinder 40 is much stronger than the electric vector potential generated by signal generator 36 through ferromagnetic ring 40. Utilizing permanent magnet 44 in elongated cylinder 43 to create the primary circumferential magnetic field keeps the electric vector potential generally linear and also provides an effective device which uses minimal electric power.

### First Embodiment (FIG. 4)

The first embodiment is a way to produce a novel field to penetrate substances. It involves a circumferentially magnetized ferromagnetic cylinder 60 immersed in or next to a magnetic substance which can be air, fluid, a human body, a chemical process, or anything that can sustain circumferential magnetization in the shape of a cylinder and a linear electric vector potential. Even outer space can probably sustain this electromagnetic configuration since space has neutrons which are magnetic or alternatively has the necessary qualities to conduct light through electromagnetic propagation. FIG. 4 illustrates this concept by showing circumferential magnetization in air 70, in secondary ferromagnetic material 66, and in living tissue 72.

FIG. 4 also illustrates the concept of making linear electric vector potential 64 more linear by providing secondary ferromagnetic cylinder 66 which becomes circumferentially magnetized 68 as linear electric vector potential 64 passes through it, in turn, making the linear electric vector potential more linear, giving it greater penetration into substances. Gradually, the ferromagnetic material will tend to align within the ferromagnetic cylinder, creating the desired circumferential magnetization within the ferromagnetic cylinder. While I believe that this occurs as the natural fluctuations in the ferromagnetic material tend to align with field induced by the magnetic loop, and I believe that, because a cylindrical magnetization has no magnetic poles, a slight magnetization force can cause a gradual, and strong magnetization within the pipe, I do not wish to be bound by this belief.

Since the electric vector potential 32 (in FIG. 3) is generally not completely linear, except when the ratio of length to wall thickness approaches infinity, linear should be understood as non-circular, or as an increasing elongation of elliptical shape 33, which gives the ability to project an electric vector potential deeper into a substance. Alternatively, it might be viewed as that part of the electric vector potential map which is generally linear 32, in which case it could be described as a linear electric field rather than a cylindrical magnetic field.

Thus, many embodiments of this invention might be envisioned in terms of generally linear electric vector potential with no mention of the magnetic component, or as circumferential magnetization of a cylindrical shape without mention of an electric vector potential.

Means of inducing such a magnetized cylinder can also vary. While the first embodiment describes simply a circumferentially magnetized ferromagnetic cylinder, we might insert a magnet into a gap in the ferromagnetic cylinder. We might wind a coil of magnet wire around the wall in an axial direction to drive current flux in a circumferential direction. This is less efficient that using permanent magnet, but allows higher magnitude field modulation. We might cause an electrical current to flow through the metal of the cylinder in an axial direction, which will induce the circumferential magnetic current in the ferromagnetic material. Alternatively, we might run electrical current axially through a non ferromagnetic conductive cylinder. The electric current would again create the circumferential magnetic current, though not so powerfully or efficiently. We might even create a cylindrical electret with an axial electrical charge, which would induce a cylindrical magnetic vector potential, which could then propagate into a substance and induce a linear electrical vector potential. Note that, in the last two embodiments, the substance carrying the magnetic flux is air, space, or other non ferromagnetic materials.

### Second Embodiment FIG. 5

The second embodiment as illustrated in FIG. 5 shows a circumferentially magnetized ferromagnetic cylinder to generate linear electric vector potential 42 with associated propagating magnetized cylinder symbolized by circle 58. This is similar to FIG. 4 except that potential 42 and magnetized cylinder 58 are used as a carrier. The first thing it carries is a signal 36 which is induced by magnetic toroid 40, which modulates the strength and perhaps the linearity of linear electric vector potential and strength of magnetized cylinder 58. The second way it is used as a carrier is by inducing photons 52 in linear electric vector potential 42, or reflecting photons with mirror 54. I do not understand the interaction between the photons and the linear electric vector potential except to observe that it makes the resulting therapy device more effective.

### Conclusions Ramifications and Scope

Although the above description contains many details, these should not be construed as limiting the scope of the invention, but merely as providing illustrations of some of the presently preferred embodiments. Other embodiments are possible, such as the following:

The term ring is used to indicate a generally toroidal shape with the principal consideration being that the ferromagnetic material makes a complete magnetic circuit around a hole, generally filled with air or other substance of low permeability. Similarly, the terms cylinder and cylindrical are used to indicate shape like a pipe or an elongated ring. Again, the principal consideration being that the ferromagnetic material makes a complete magnetic circuit around a hole of low permeability. Devices can easily be made with, for instance, rings that are not round, and cylinders that have variable wall thickness or non-circular cross section.

These principles can be applied broadly to therapy devices and process equipment of many kinds, for example, holding a linear electric field during crystal growing, the crystal might grow faster or differently as elements are held in alignment in the liquid state, and exotic new crystals might be possible. Biochemical processes might be catalyzed or prevented. New semiconductor effects might be developed which depend on this linear electric vector potential arrangement. Power generation may be possible in unique new ways with cold fusion, or Zero Point devices. These modulated linear electric potentials might be used to reduce smog, create new elements, accelerate crop growth, or develop new strains of crops. New communication methods might also utilize this linear electric potential as a broadcast carrier method.

The scope of this invention should be determined by the appended claims and their legal equivalents, rather than by the examples given.

There are many means to create magnetic currents in a cylinder or a ring. While the described embodiments use a permanent magnet for the cylinder and a toroidally wound coil for the ring, a wide variety of means might be employed to create or modulate the cylindrical magnetic field and linear electric vector potential. “Toroidally wrapped” is intended here to mean wrapped around some part of the cross section of a ring, and not limited to uniformly distributed windings around the ring.

While the intention is that the preferred embodiment induce therapeutic signals into a body for therapy, the specific signals to be induced can be highly varied. Prior art patents of this inventor such as U.S. Pat. No. 6,461,316, U.S. Pat. No. 6,770,042, and U.S. Pat. No. 7,419,474 provide examples of the sorts of signals that might be induced, though a host of other signals including sine and square waves and pulses might also be employed.

The cylindrical magnetic field might be projected into water such as a bath tub, hot tub, pool, lake or ocean, or into air, plants or soil, or any other substance, including etheric substances like fields of neutrons, photons, gravitational fields, or into the ether of space.

While in the first embodiment a 630 nm LED is utilized to inject photons into the cylindrical magnetic field, any light source or electromagnetic source might be used. Similarly, while a front surface aluminum mirror is to be used in the described embodiment, any reflective surface can be used depending on the frequency of light to be reflected or absorbed.

What is claimed is:

1. A method for inducing a cylindrical magnetic field in a substance, comprising:
a circumferentially magnetized cylinder with cylinder wall, and a substance positioned axially to said cylinder,
wherein said field is induced in said substance, and
wherein the length of said cylinder is at least twice the thickness of said cylinder wall.

2. The invention of claim 1 wherein means to magnetize said circumferentially magnetized cylinder is selected from: electromagnetic coil, permanent magnet, and a second cylindrical magnetic field.

3. The invention of claim 1 wherein said cylinder comprises ferromagnetic material.

4. The invention of claim 1 also comprising a means of adding photons into said cylindrical magnetic field.

5. The invention of claim 1 comprising a means of reflecting photons parallel to length of said magnetic cylinder.

6. The invention of claim 1 wherein said substance comprises living tissue.

7. The invention of claim 1 further comprising a method for modulating said field, comprising:
signal generating means, magnet wire, and ferromagnetic ring
wherein said magnet wire is toroidally wrapped around said ring, and said cylindrical magnetic field passes through said ring, wherein a signal from said signal generating means is applied to said magnet wire, thereby modulating said field.

9. A method for modulating a cylindrical magnetic field; comprising:
cylindrical magnetic field, signal generating means, signal produced by said signal generating means, magnet wire, and ferromagnetic ring,
wherein said magnet wire is toroidally wrapped around said ring, and said field passes through said ring, wherein said signal from said signal generating means is applied to said magnet wire thereby modulating said field.

10. An electrical vector potential projector comprising:
a circumferentially magnetized cylinder, a linear electric vector potential produced by said cylinder, and a substance,
wherein said cylinder projects said linear electric vector potential into said substance.

11. The invention of claim 10 also comprising:
signal generating means and linear electric vector potential modulating means
wherein said modulating means modulates said linear electric vector potential according to the signal of said signal generating means.
